(19) United States
(12) Patent Application Publication
(10) Pub. No.: US 2005/0027332 A1
(43) Pub. Date: Feb. 3, 2005

(54) Implanted autonomic energy source

(76) Inventors: Zohar Avrahami, Rehovot (IL); Yossi Gross, Moshav Mazor (IL)

Correspondence Address: Martin Moynihan;c/o ANTHONY CASTORINA, SUITE 207, 2001 JEFFERSON DAVIS HIGHWAY, ARLINGTON, VA, 22202, US

(21) Appl. No.: 10/891,156

(22) Filed: Jul. 15, 2004

(30) Foreign Application Priority Data
Jul. 30, 2003 (IL) 157173

(51) Int. Cl. A61N 1/08

(52) U.S. Cl. 607/61

(57) ABSTRACT

Implanted Autonomic Energy source which comprises of at least one portion of a living organism that provides useful energy responsive to electrical stimulation, at least one energy converter that receives the said useful energy and converts it into electrical energy and electrical stimulation means; wherein at least some of the said useful energy is converted into electrical energy, energizing the said electrical stimulation means, provided that the said electrical energy is at least sufficient to energize the stimulation means, thus forming an electro-biological positive feedback loop that provides an autonomic useful energy source, basically been energized by the living organism metabolism.

This application claims the benefit of priority from Israel Patent Application No. 157173, filed Jul. 30, 2003.

### FIELD OF THE INVENTION

The present invention is related to medical devices. More specifically, the present invention is related to implanted autonomic energy source for supplying useful energy to implanted means.

### BACKGROUND OF THE INVENTION

Implantable devices that require power sources are well known in the art; their power sources are often used to provide power for sensing, control, tissue stimulation, drug dispensing, external communication, and other necessary functions such as heart assisting devices, pumps and neurological stimulators. Some of the prior art improvements in such implanted power sources are discussed below by reference to several United States patents; the entire disclosure of each of these United States patents is hereby incorporated by reference into this specification.

Trumble in U.S. Pat. No. 5,479,946 provides in the background description a comprehensive review of the various prior attempts of hustling muscle energy for energizing implanted medical devices. Trumble's invention relates to converting muscle power into controlled mechanical power for direct operation of mechanical devices such as cardiac assisting devices. Snaper; Alvin A.; Gelbaum; and Bernard R.; in U.S. Pat. No. 5,431,694 further describe a muscle operated piezoelectric generator in which a piezoelectric generator in the form of a flexible sheet of poled polyvinylidene fluoride structurally is attached in surface-to-surface contiguity with a skeletal member, which flexes with negligible elongation of its surface, is connected in circuit with a power consuming device such as a pacemaker, to a rectifier, and to a power storage device such as a condenser or battery. Flaherty; J. Christopher in U.S. Pat. No. 5,810,015 provides a survey of power supply methods for implanted devices, while providing a muscle powered pressure fluidic engine for powering implanted devices. MacDonald in U.S. Pat. No. 6,640,137 and Weijand, et al. in U.S. Pat. No. 6,470,212 demonstrate the need for autonomic power source and provide an electrical power supply method for implanted devices, based on exploiting temperature gradients within the living organism to energize a thermoelectric module. However said method is very limited in terms of the available useful power (about 0.1 mW at temperature difference of 2 degrees and perfect thermal contacts through which all the thermal input energy must flow) and is dependent on the living body conditions.

From the above mentioned references the following observations with relevance to the present invention are made:
Some specific muscles of a living body may be used for providing mechanical energy to an energy-converting device. The skeletal muscles are a good example and may provide mechanical powers in the order of Watt.
Devices may be mechanically coupled to muscles in order to receive muscle mechanical energy and convert it into useful electrical energy by various methods such as piezoelectricity and magnetoelectricity.
The said electrical energy may be stored by means such as rechargeable batteries or capacitors.
Muscles through medical operational procedures may be modified including detaching, slicing and attaching while maintaining muscle functionality.
Electrical pulses may stimulate muscles.
Muscles may be trained and conditioned to resist fatigue, for example in order to utilize skeletal muscle for long-term operation, a conditioning scheme is needed to convert these fibers from glycolytic to oxidative metabolism.
Total internal muscle energy converters require employment of only autonomic muscles, definitely reducing muscle selection options.
Controllable stimulated muscle energy converters require utilization of electrical source either implanted or external, which is a major drawback for implanted energy source applications.
The amount of muscle available energy depends upon its size, type and its rate of stimulation.

Clearly there is a need for autonomous power supply for powering implanted devices, which converts living body metabolic energy into controllable useful energy.

One objective of the present invention is to provide a method and device for an implanted autonomous power supply. Additional objectives will become apparent from the description of the invention.

### SUMMARY OF THE INVENTION

Definitions:

For the purpose of this invention the following definitions in addition to ordinary context, shall apply:

Electro-biological energy loop or energy loop shall mean an interconnected configuration of devices and biological organs through which energy propagates in a closed loop.

Useful energy shall mean the energy provided by a biological organ that may be hustled, controlled, stored or directed in order to perform defined work.

Electrical stimulation energy shall mean the total required electrical energy/power for stimulating the biological organ or muscle.

Living muscle organism shall mean a portion of living organism consisting a muscle tissue belonging to a living body supported by its life support biological processes.

Oscillating muscle shall mean an alternating contracting-relaxing muscle responsive to external electrical stimulation.

Excess useful energy shall mean said useful energy minus the total energy consumed by the stimulation and control means.

Converter means shall mean any device adapted to receive energy from the biological organism and convert it into some other form of useful energy. For example, a moving coil generator attached to a muscle; a piezoelectric/magnetostrictive device attached to a muscle; a piston of an hydraulic device attached to a muscle; a thermoelectric element attached to living organs; and a pressurized flexible chamber attached to a muscle.

Multi-form energy source shall mean a source of useful energy with at least two outputs with different energy forms. Example, a source with terminals providing electrical voltage and an outlet pipe that provides hydraulic power.

Electrical storage means shall mean device that accepts electrical energy, accumulates it, may adapt it and deliver it to loads. Examples—capacitor, converters, inductors and rechargeable battery.

Implanted Autonomic Energy source shall mean implantable device or devices adapted to provide energy to other implanted modules provided that it is fully energized by the living body host and do not require any external energy.

Electrical stimulation means shall mean electronic and electrode means to provide electrical pulses for stimulating the biological organ, which responsibly changes its available energy.

Sufficient useful energy shall mean the minimum amount of useful energy, taking into consideration system components efficiency, required for sustained electro-biological energy loop operation.

Control means shall mean a preprogrammed machine such as a micro-controller chip for real-time control of stimulation signals parameters, such as repetition rate, burst length, amplitude, duration and shape.

Initial stimulation shall mean the stimulation required to bring the loop into a self-sustained state. The initial stimulation energy may be provided by initially charged battery.

The invention presents a novel electro-biological energy loop, which enables to convert living organism metabolic energy into other forms of useful energy for driving implanted devices. According to the present invention, a loop consisting of:
living organism energized through metabolic processes, producing useful energy responsive to electrical stimulation;
means for stimulating said organism;
means for converting said useful energy into electrical energy which further energizes the said stimulating means.

Due to the metabolism of the living organ, it is appreciated that the electrical stimulation energy may be lower than the produced useful energy. Thus by a proper configuration a self-sustaining positive feedback electro-biological loop may be implemented.

For a non-limiting example, consider a portion of a living muscle organism stimulated by electrical pulses for repetitive contraction and relaxation. A magnet attached to the muscle moves within a coil generating electric energy at coil terminals. The electrical energy may be rectified and stored in a rechargeable battery. The battery energizes a pulse generator circuit, which provides electrical stimulation via electrodes attached to the said muscle organism. A positive feedback closed energy loop is thus created. If the generated electrical energy is greater than the required stimulating energy, muscle exhibits self-sustained oscillations for, as long as, the metabolism process provides the basic energy.

It is further appreciated that the said oscillating muscle may provide more useful energy than that required for sustaining loop oscillations; the excess useful energy may be used for driving additional devices or systems. By attaching additional converter means to said oscillating muscle, a multi-form energy source may be implemented.

As a non-limiting example, refer to the said above energy loop wherein a diaphragm pump is also mechanically coupled to the oscillating muscle, a useful hydraulic energy is then generated in addition to the electrical source.

Loop oscillations are preferably initiated by using an initially charged, electrical storage means, such as an initially charged rechargeable battery or charged capacitor. The initial energy should be large enough to ensure loop steady state oscillations.

As known, oscillating muscle is susceptible to fatigue. Muscle fatigue effects may be minimized by muscle selection, preferably smooth muscles but not limited to, conditioning training and by controlled regime and scheduling stimulation such as allowing relaxation time intervals sufficient for organism recovery. Preferably by controlling muscle stimulation in a close loop, in accordance with sensible characteristic muscle fatigue products and or in accordance with the power consumption characteristic pattern using either an adaptive or a preprogrammed scheduler for avoiding a too high muscle stress.

It is further appreciated that said muscle might be comprised of multiple segments of muscles connected in certain possible configurations and stimulated separately to provide the desired motion or stress. For a non-limiting example, two muscles may be connected to operate in a substantial “push-pull” configuration, where stimulation is alternately applied to each of the muscles.

The present invention, Implanted Autonomic Energy source, comprising of: at least one portion of a living organism that provides useful energy responsive to electrical stimulation; at least one energy converter that receives the said useful energy and converts it into electrical energy and electrical stimulation means; wherein at least some of the said useful energy is converted into electrical energy, energizing the said electrical stimulation means, provided that the said electrical energy is at least sufficient to energize the stimulation means, thus forming an electro-biological positive feedback loop that provides an autonomic useful energy source, basically been energized by the living organism metabolism.

According to one aspect of the present invention, the said portion of living organism comprises at least one portion of muscle organism, which upon electrical stimulation provides mechanical energy.

According to another aspect of the present invention, the said energy converter comprises a mechanical to electrical power converter, mechanically driven by the said muscle organism.

According to a further another aspect of the present invention, additional energy converters may be used to convert the said living organism useful energy into other forms of useful energy such as mechanical energy, hydraulic energy, electrical energy and thermal potential energy.

The present invention is also a method of generating useful energy within a living body. The method comprises the steps of:
selecting a portion of a living biological organism wherein said biological organism is capable of at least providing sufficient useful energy responsive to electrical stimulation and may be repetitively stimulated without substantially interfering with the hosting biological system;
attaching to said organism electrical stimulating means;
coupling to said organism at least one energy to electrical converter means capable of energizing the said stimulation means;
feeding via control means said converted electrical energy to the said stimulating means;
providing initial stimulation sufficient for producing enough electrical energy for sustained electro-biological loop operation.

Another aspect of the said method of generating useful energy within a living body, wherein the method comprising the steps of:
Detaching at least a portion of a living biological organism while maintaining its metabolic interaction with the living body, wherein said biological organism is capable of at least providing sufficient useful energy responsive to electrical stimulation;
attaching to said organism electrical stimulating means;
coupling to said organism at least one energy to electrical converter means capable of energizing the said stimulation means;
feeding via control means said converted electrical energy to the said stimulating means;
providing initial stimulation sufficient for producing enough electrical energy for sustained electro-biological loop operation.
Preferably, the said portion of living biological organism is a muscle organism.

### BRIEF DESCRIPTION OF THE DRAWINGS

In the accompanying drawings, the preferred embodiment of the invention and preferred methods of practicing the invention are illustrated in which:

FIG. 1 is a schematic representation showing an Electro-Biological Loop.

FIG. 2 is schematic representation showing a muscle organism electro-biological loop.

FIG. 3 is schematic representation showing an example of an electro-biological loop configured as a multi energy form source.

FIG. 4 is a schematic representation showing two muscles “push pull” configuration electro-biological loop.

FIG. 5 is a schematic representation showing an embodiment of a piezoelectric/magnetostrictive muscle energy converter.

### DESCRIPTION OF THE PREFERRED EMBODIMENT

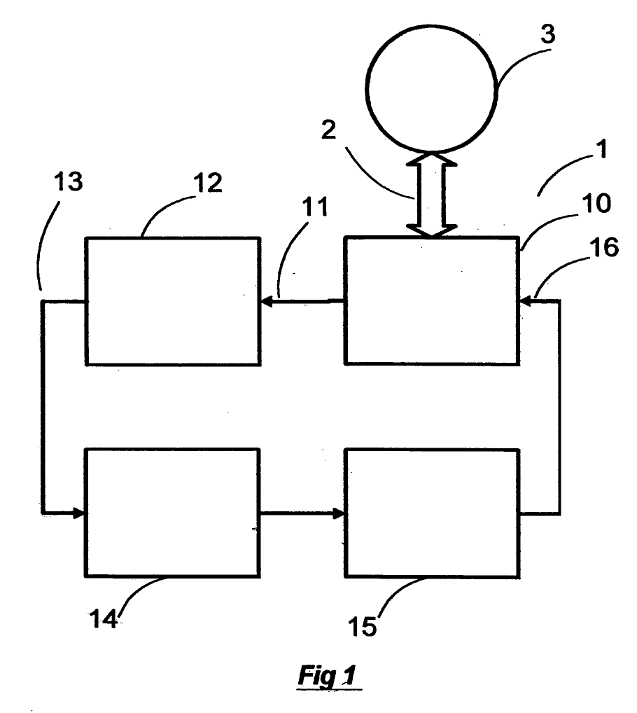

Reference is made to FIG. 1 illustrating the basic electro-biological energy loop 1. The loop 1 is embedded in a living body organism 3, which provides energy and life survival conditions to a portion of biological organism adapted for providing useful energy 10. The said biological organism 10 is interacting with the living body 3 via circulatory system and all inherent life sustaining channels 2. It is appreciated that living body 3 provides basic energy through the metabolic process. It is further appreciated that said biological organism is adapted to provide useful energy 11 responsive to stimulation. An energy converter that converts useful energy into electrical energy 12 is attached to the biological organism 10, to receive useful energy 11. At converter output terminals, electrical energy 13 is available, processed and stored by electrical energy storage means 14. The electrical storage means 14, provide the energy to stimulation means 15 through electrical connection. The stimulation means 15 are further connected to said biological organism 10, via conducting and electrode means 16, to provide the said stimulation, thus closing an electro-biological energy loop 1. The electro-biological energy loop 1, converts the metabolic energy provided by the living body 3 into useful energy form that can be further employed for energizing additional means implanted in the living body (not shown). It is appreciated that for sustained operation the loop energy ratio, i.e. the ratio of the energy available at the electrical storage output terminals to the energy consumed by the stimulation means, should be greater than 1.

Although biological organism may be of different types and may provide different forms of useful energy such as thermal energy or chemical potential energy, due to secretion of chemical compounds, it is preferably according to present invention to select a portion of muscle organism, which provides mechanical useful energy responsive to electrical stimulation. Known in the prior art (U.S. Pat. No. 5,479,946) it is possible to detach a portion of a muscle from at least one tendon and attach it to other structures while maintaining its functionality in terms of metabolism and other life supporting processes.

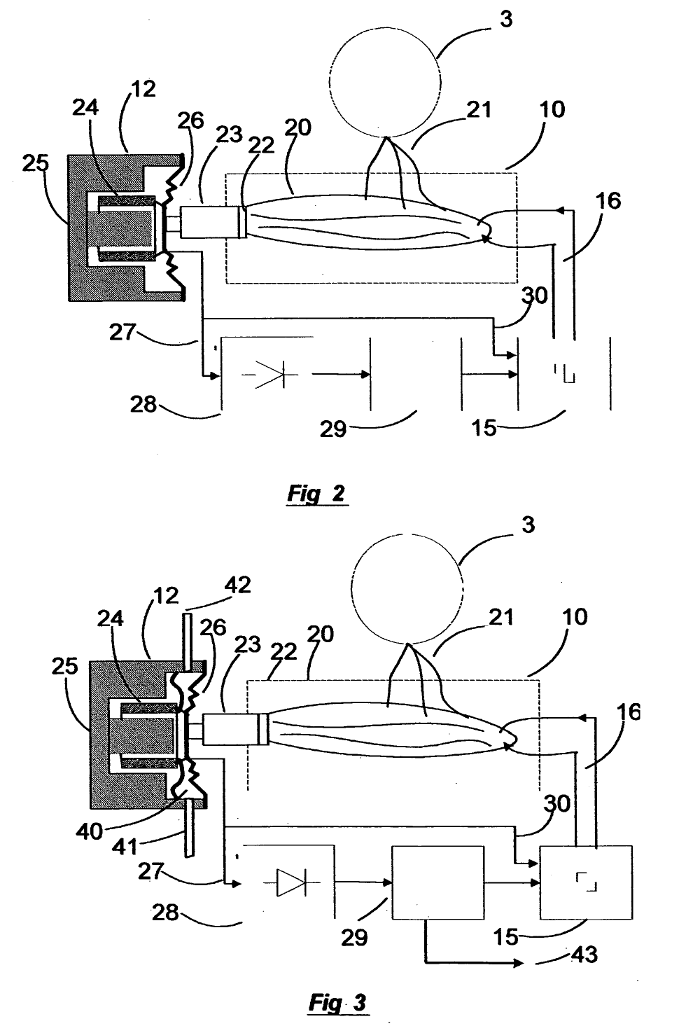

Reference is made to FIG. 2 wherein a muscle organism electro-biological loop is illustrated. A portion of a muscle organism 20, having its life supporting and metabolic channels 21 to its living body 3, is attached by hooking means 22 and a tension transfer, preferably auto adjusted consisting for example a spring and ratchet mechanism, member 23 to a moving coil 24, exerting linear force while contracting. The moving coil 24 is supported by a preloaded structure 26 which exerts force on the moving coil in substantially opposite direction to muscle contraction force so that to allow the moving coil 24 to move upon muscle contraction in the contraction direction and move in the opposite direction upon muscle relaxation. The moving coil 24 moves within a magnetic field created by permanent magnet structure 25 such as for example the well-known configuration of a moving coil loudspeaker. It is appreciated that upon coil movement electrical power is available at coil terminals 27. The AC power is further processed and rectified by electronic circuit 28 known to any professional in the field, preferably by using CMOS switches to increase efficiency. The rectified electrical energy is stored in the electrical storage means 29, preferably implemented by rechargeable battery, but may also be implemented by super-capacitors. The said stored electrical energy is connected via conductors to the electrical stimulating means 29. The output of the electrical stimulating means 29, consists of pulses preferably burst of pulses applied to the muscle organism by electrodes 16 for stimulating muscle contraction, thus closing the said electro-biological energy loop 1. Muscle electrical stimulation is widely known and published in the literature (for example see U.S. Pat. No. 5,479,946 and its references), typical pulses are of 0.5V to 10V and currents in the range of 0.1 to 100 mA at burst duration of 0.05 to 1 second. It is preferably to stimulate the muscle by applying electrodes 16 to or near the motor nerve, but they may be connected to other locations on the muscle organism as well. The electrical stimulating means 15 is implemented by ordinary known electronic pulse circuitry, preferably by employing CMOS technology to increase efficiency. It is further recommended to synchronize the stimulation pulses to muscle contraction, for example by using the output voltage of the moving coil as synchronizing signal 30 or by employing other sensors attached to the muscle organism (not shown). It is further appreciated that the loop may be designed to operate at its natural mechanical resonance for higher efficiency. The repetitive oscillating muscle is susceptible to fatigue effects. It is already known in the art that muscles, preferable but not necessarily smooth muscles, may be trained in order to increase their fatigue resistance as described also in U.S. Pat. No. 5,479,946. It is therefore preferably to use a preprogrammed machine as a stimulation controller incorporated in the said stimulation means 15, which may be implemented by CMOS micro controller chip, and which accepts signals from biosensors attached to the muscle organism, indicative of muscle fatigue by-products, such as any combination of: blood oxidation level, muscle temperature, or concentration of: creatine phosphate, adenosine triphosphate (ATP), lactate, glycogen content, myosin ATPase, and succinate dehydrogenase (SDH), and to regulate in accordance to a preprogrammed control algorithm the stimulation parameters including stimulation scheduling, adaptive scheduling per power consumption pattern, rate and relaxation periods for minimizing the muscle over-stress.

It is further appreciated that the requirement of a loop energy ratio greater than 1 is practically achievable by a proper design. For example, consider the following energy budget:
Muscle useful power—5 W
(Trumble in U.S. Pat. No. 5,479,946 describes measurements conducted with canine latissimus dorsi muscles (LD), accordingly “Six canine LD muscles were evaluated using the MAPP/mock loop system. The muscles were not mobilized, thereby preserving their collateral circulation. A customized stimulating lead was implanted over the thoracodorsal nerve. The tendinous insertion of the LD to the humerus was transected and connected to the MAPP. Preload was adjusted to return the LD to its in-situ length, and pulse trains were delivered at a rate of 60 per minute. Each burst stimulus consisted of 11 pulses delivered at a rate of 43 Hz. Under these conditions, average peak power levels reached 10.75 watts, while mean power during shortening exceeded 5.5 watts. This level of performance easily exceeds that of many previous studies, and reflects the improved muscle perfusion that results when the LD is left in situ and not mobilized from the chest wall on a single vascular pedicle. These data are especially encouraging since the average mass of these muscles was only 200 grams”.)
Mechanical to electrical converter efficiency—more than 0.5, typical efficiency of moving coil generator.
Electrical rectifying and processing efficiency—more than 0.8, typical efficiency of low voltage CMOS converters.
Electrical stimulation means efficiency—more than 0.9 typical efficiency of low frequency CMOS pulse circuitry.
Muscle stimulation power—less than 0.1 W (assume typical pulses of 0.2 sec. burst duration at 1V and 5 mA and burst rate of 60 bursts per minute resulting in 1 mW power).

Total loop energy ratio in such a typical case, is 18 greater than 1. The excess useful energy, about 4.5 watts, is in this case more than 1 W.

As already mentioned above, the useful energy to electrical converter is essential for providing at least the energy consumed by the control and stimulating means, however it is appreciated that the converter may produce excess electrical power to energize other power consuming devices such as pacemakers, implanted sensors and implanted bio-controllers. In some applications it is preferably to use other forms of energy like mechanical energy for powering implanted pumps or hydraulic energy form that may be conveyed through pipes. Reference is made to FIG. 3 in which an electro-biological loop is, for example, adapted to provide hydraulically and electrical energy, i.e. a multi-energy form source. The muscle organism 10 is connected to a preloaded diaphragm 26 constituting a part of a diaphragm based pump mechanism 40. Upon muscle contraction the diaphragm is pulled towards expanding pump cell volume, while during muscle relaxation preload force pushes back the diaphragm providing pump cell compression. A moving coil, free to move in a magnetic field similar to the illustration given in FIG. 2, is attached to the moving diaphragm, tracking diaphragm oscillatory motion. Electrical energy is generated at coil terminals as described above. A two-form energy source is therefore provided where the electrical output power is available at terminals 43 and hydraulic power is available at tube outlets 41 and 42. It is further appreciated that this conceptual example shows only the basic concept of the invention and may be employed, designed and implemented in various different configurations by professionals in the field.

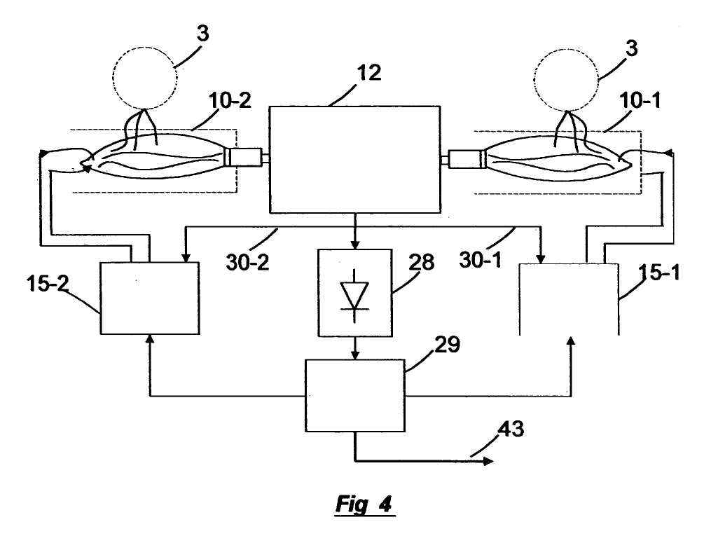

Reference is made to FIG. 4 in which an electro-biological loop composed of two muscle organisms 10-1 and 10-2, in a “push pull” configuration is illustrated. Two individually stimulated said muscle organisms are connected to a mechanical to electrical energy converter means 12. The electrical energy is processed and stored as described above. The stored electrical energy energizes both stimulating means 15-1 and 15-2. The said stimulating means provide stimulation pulses to their corresponding muscle organism in synchronization to cause alternating contractions resulting in a “push-pull” force exerted on the mechanical to electrical converter means 12.

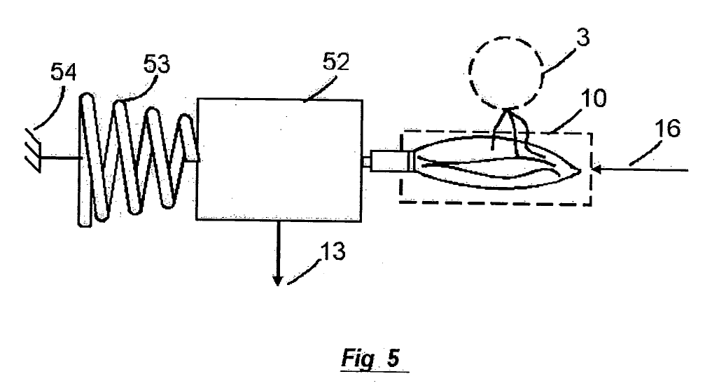

Alternatively, in applications where it is preferably to use muscle tension rather than motion, piezoelectric or magnetostrictive converter means may be employed. Reference is made to FIG. 5 in which an electro-biological loop with piezoelectric/magnetostrictive energy converter 52, is schematically shown. In this configuration, practically no physical motion is involved which in some cases is beneficial in terms of repetition rate, muscle efficiency and fatigue. A piezoelectric device 52 is attached to the muscle organism 10. Upon muscle contractions responsive to muscle stimulations 16, strain is introduced in the piezoelectric element generating electrical voltage at its terminals 13. The electrical energy is further processed and stored as has been explained above. Although utilization of piezoelectric device is well known (example—U.S. Pat. No. 5,431,694), the claimed innovation in the present invention is the employment of piezoelectric converters in electro-biology energy loops. Similarly, instead of a piezoelectric device, it is possible to employ a magnetostrictive device to convert strain into electrical current that is further processed, and stored as electrical energy.

U.S. Pat. No. 5,479,946 describes in details muscle electrical stimulation for attaining mechanical energy at low muscle fatigue and is hereby incorporated as reference.

Although the invention has been described in detail in the foregoing embodiments for the purpose of illustration, it is to be understood that such detail is solely for that purpose and that variations can be made therein by those skilled in the art without departing from the spirit and scope of the invention except as it may be described by the following claims.

1. An electro-biological energy loop comprising:
at least one portion of a living biological organism, belonging to a living body which provides it with the metabolism and the life supporting processes, adapted to provide useful energy responsive to electrical stimulation;
at least one energy converter device that is adapted to accept said useful energy and to, at least, convert it into one electrical energy output; and
stimulating means, energized by said energy converter, adapted to provide stimulation to said living biological organism;
connected in a closed loop configuration wherein the said useful energy is coupled to energize the said, at least one, energy converter device; the output of said energy converter is connected to the said stimulating means, providing it with electrical energy; the output of said stimulating means is connected to said living organism providing it with electrical stimulation, which further provides the said useful energy responsive to said stimulation.

2. An electro-biological energy loop described in claim 1, wherein the said portion a living biological organism constitutes at least one portion of a muscle organism adapted to provide useful mechanical energy responsive to muscle electrical stimulation.

3. An electro-biological energy loop described in claim 2, wherein the said portion a living biological organism constitutes at least two portions of muscle organisms adapted to provide useful mechanical energy by exerting bi-directional force on the said at least one energy converter, responsive to muscles electrical stimulations.

4. An electro-biological energy loop described in claim 1, wherein the energy available at the output of said energy converter is greater than the energy consumed by the said stimulation means thus enabling sustained loop operation.

5. An electro-biological energy loop described in claim 1, wherein the loop consists energy storage means.

6. An electro-biological energy loop according to claim 5, wherein the said storage means consists at least one member selected from the group: rechargeable battery, capacitor including super capacitors, pressurized reservoir, fly wheel and inductor.

7. An electro-biological energy loop according to claim 1, wherein the said energy converter device consists at least one member selected from the group: moving coil, moving magnet, piezoelectric, magnetostrictive and thermoelectric.

8. An electro-biological energy loop according to claim 2, wherein the said stimulation is provided in synchronization with said muscle contraction.

9. An implanted energy source for providing useful energy to at least one implanted functional device comprising of at least one electro-biological loop as described in claim 1.

10. An implanted energy source according to claim 9, wherein the said at least one functional device is selected from the list: pacemaker, defibrillator, pump, electrical stimulator, drug delivery device, telemetry device, heart assisting device and sensor.

11. An implanted energy source according to claim 9, wherein the output useful energy forms constitutes at least one form selected from the list: electrical, mechanical and hydraulic.

12. An implanted energy source according to claim 9, wherein the initial energy, required for sustained said electro-biological energy loop operation, is provided by the initially stored energy in the said energy storage means described in claims 5 and 6.

13. An electro-biological energy loop described in claim 1, wherein said stimulating means consists a preprogrammed controller that controls at least one stimulation parameter in accordance to at least one input control signal.

14. An electro-biological energy loop described in claim 13, wherein, the said at least one stimulation parameter is selected from the list: repetition rate, burst repletion rate, pulse duration, pulse interval, pulse shape, pulse amplitude, pulse timing, burst length, timing on/off periods and burst modulation.

15. An electro-biological energy loop described in claim 13, wherein the at least one input control signal is derived by sensing of at least one member selected from the list: energy consumption, output voltage, output current, muscle movement, temperature, elapse time, real time and specific substance concentration.

16. An electro-biological energy loop described in claim 13, wherein said preprogram include a time scheduler routine.

17. An electro-biological energy loop described in claim 16, wherein said time scheduler routine includes an adaptive algorithm driven by at least the power consumption.

18. An electro-biological energy loop according to claim 2 adapted to substantially operate at a loop natural resonance frequency.

19. A method of generating useful energy within a living body, wherein the method comprises the steps of:
a. selecting a portion of living biological organism while maintaining its metabolic interaction with the living body wherein said biological organism is capable of at least to providing useful energy responsive to electrical stimulation;
b. attaching to said organism stimulating means;
c. coupling to said organism at least one converter means of useful energy to electrical energy capable of energizing the said stimulation means;
d. feeding the said converted electrical energy to the said stimulating means.

20. A method of generating useful energy within a living body according to claim 19, wherein the said biological organism is muscle.

21. A method of generating useful energy within a living body according to claim 19, wherein step 1 includes detaching a portion of said living organism.

22. A method of generating useful energy within a living body according to claim 19, wherein a fifth step is added: 5. providing initial electrical energy for sustained said electro-biological energy loop operation.
